(19) United States
(12) Patent Application Publication
(10) Pub. No.: US 2008/0044351 A1
(43) Pub. Date: Feb. 21, 2008

(54) METHOD AND PROCEDURE TO DETECT ORAL TISSUES WITH HIGH-RISK MOLECULAR PROFILES

(76) Inventor: Xin Ye, Chicago, IL (US)

Correspondence Address: Xin Ye, 6029 S. Kimbark Ave. #1, Chicago, IL, 60637, US

(21) Appl. No.: 11/464,536

(22) Filed: Aug. 15, 2006

(51) Int. Cl. A61K 49/00

(52) U.S. Cl. 424/9.1; 424/53

(57) ABSTRACT

A diagnostic method for detecting high risk oral tissue which due to chromosome allelic loss and may have developed or will potentially develop into carcinoma in situ and carcinoma by using a staining agent. A set of questionnaire that identifies high-risk individual and high-risk lesions and helps to reduce false positive for the diagnosis of oral tissue with high-risk molecular profile. A recording sheet with oral cavity diagram that helps to record the examination results more accurately and reduce the examination errors. This improved method has a broad application that can be used to screen the oral tissues with high risk molecular profile from hyperplasia, dysplasia, erythroplasia, leukoplasia, leukoerythroplasia, erythroleukoplasia, verrucous lesion, ulcerative lesion, carcinoma in situ, and carcinoma.

### REFERENCE CITED

U.S. Patent Document

| | | |
|---|---|---|
| 4321251 | Mar. 23, 1982 | Mashberg |
| 6417003 | Jul. 9, 2002 | Cipriani et. al. |

### Other Publications

Guo, Clin. Can. Res. Vo. 7, 2001, pp. 1963-1968.
Mashberg, Cancer. Vol. 37, 1976, pp. 2149-2157.
Mashberg, C A Can. J. Clin. Vol. 45, 1995, pp. 328-251.
Kujan. J. Den. Edu. Vol. 69, 2005, pp. 255-265.
Epstein. Oral Sur. Oral Med. Oral Path. Vol. 95, 2003, pp. 45-50.
Gray. W. Midland DES Rep. 2000, pp. 1-41.
CDC Oral Can. Background Papers, Chpt. III, IV, V, IX.
FDI Policy Statement, 1998

### DESCRIPTION

### Brief Summary of the Invention

This invention relates to improved methods and procedure of screening patients, using an oral staining agent, toluidine blue, for high risk oral tissue which is due to chromosome allelic loss (or, Loss of Heterozygosity, LOH) and may have developed or will potentially develop into carcinoma in situ and carcinoma.

In another respect, the invention relates to a kit for use in screening patients for high risk hyperplasia, dysplasia, erythroplasia, leukoplasia, leukoerythroplasia, erythroleukoplasia, verrucous lesion, ulcerative lesion, carcinoma in situ, and carcinoma.

In yet another respect, the invention relates to a kit including a questionnaire sheet to identify high-risk individuals and reduce the false positive, and a recording sheet to avoid some examination errors in screening patients for high risk oral tissue. In the mean time, the kit comes with packaged form and would be convenient for clinicians and dentists to perform routine office-visit examination.

### BACKGROUND OF THE INVENTION

Oral cancer progression from benign to malignant is a genetic process, which occurred at molecular level at first, then occurred at cellular level, and eventually occurred at clinical level and can be detected by histopathology diagnosis. The transition time from dysplasia to malignancy may take up to 39 years. Histopathology diagnosis reflects cellular changes that are visibly apparent but does not necessary predict biologic behavior, which was determined at molecular level.

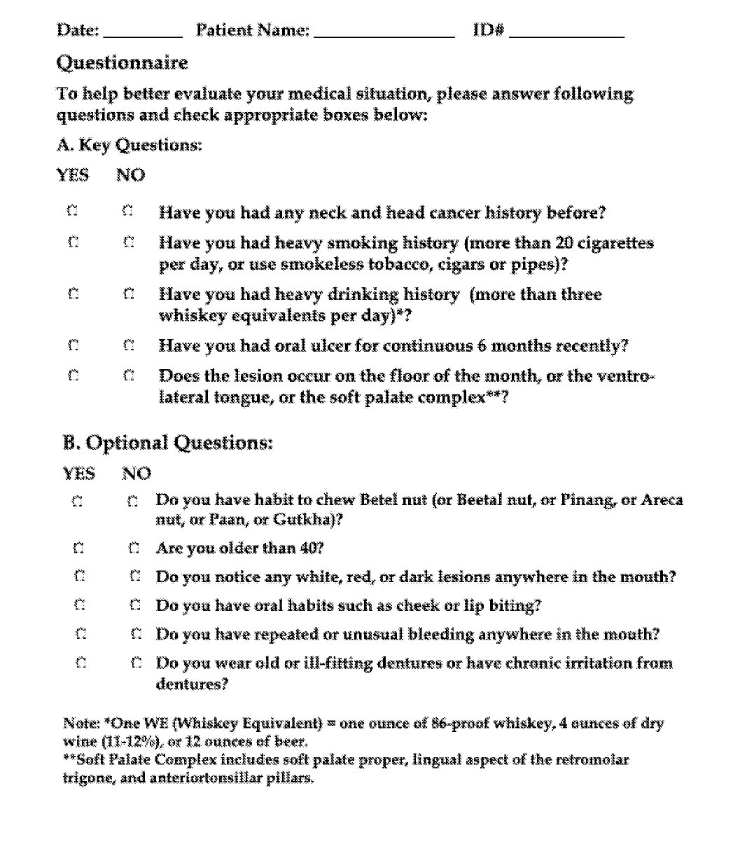

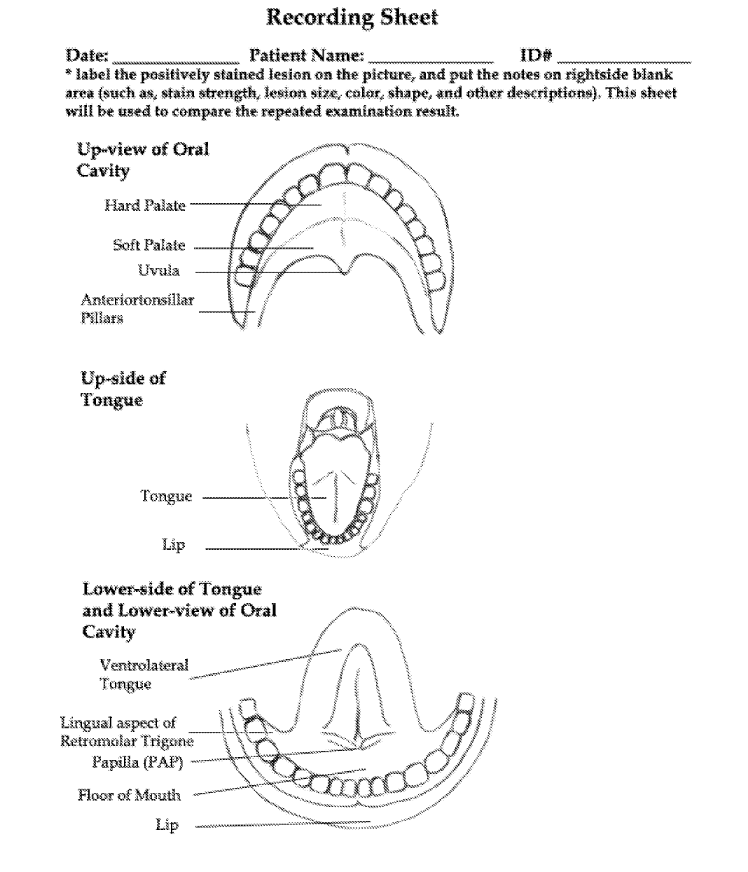

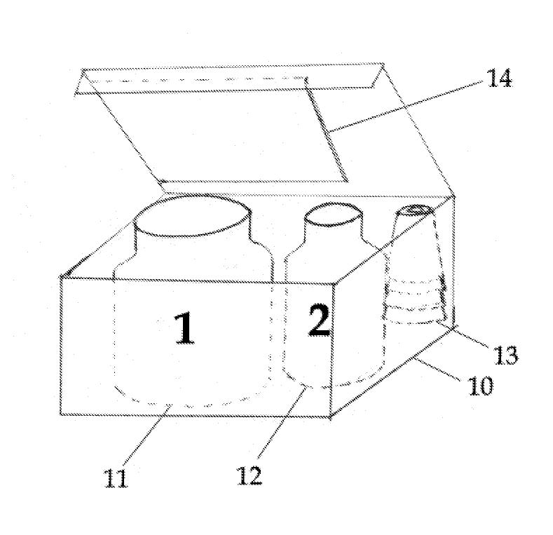

Chromosome allelic loss (or Loss of Heterozygosity, LOH) is a predictive way to determine cancer risk for oral premalignant lesions, especially the LOH contains any known or presumptive tumor suppressor genes. Some LOH that have already been studied and found to have impact for cancer development include: 3p, 4q, 8p, 9p, 11q, 13q and 17p, etc. The risk of progression to cancer is low when there is no genetic change. The risk would significantly increase if there is LOH occur, especially there are multiple LOH or there is loss of critical chromosome regions (such as 3p14 and 9p21). For example, high-risk lesion, as indicated by LOH, may progress to cancer over a 5-year period in up to 50% of cases, while low-risk lesions progress to cancer in only 2% of cases. FIG. 1 showed a molecular model of carcinogenesis.

Toluidine blue (tolonium chloride) is a metachromatic dye that stains mitochondrial DNA, cells with greater-than-normal DNA content, or altered DNA in dysplatic and malignant cells. The stained lesions usually have changed high-risk molecular profile, LOH.

Studies have showed that toluidine blue-positive lesions had a higher frequency of LOH than toluidine blue-negative lesions. Sometime the toluidine blue-positive lesions even contain a loss of multiple arms of chromosome, a pattern that has been associated with increased cancer risk. Toluidine blue staining can be used to help identify oral premalignant lesions with increased LOH and increased cancer risk.

Oral squamous cell carcinoma (SCC) stains strongly with this dye, while oral dysplasia has much less consistent results, which contributed a lot for the false positive results. Dysplasia staining also differs in the intensity, with some strong, some weak, and some equivocal. The possibility for this variation is that there are molecular differences in dysplasia that are toluidine blue-positive and toluidine blue-negative, or toluidine blue-equivocal (weakly positive). Toluidine blue-positive lesions tend to contain high-risk molecular profile. Even the lesions appeared normal under microscope but detected by toluidine blue as positive will still contain the critical clonal changes that are necessary for cancer progression.

There are also risk factors for oral cancer, such as tobacco and alcohol use, may result in broad areas of change in the oral mucosa. Molecular changes may be occurred in these areas before cellular phenotypic changes become detectable. Today, the 5-year survival rate for oral squamous cell carcinoma (SCC) still remained at 50%, without much improvement from past decades, because many oral SCCs are not diagnosed or treated until late stage. Some reasons for not effectively detecting early oral cancer are: (1) fail to identify individuals at highest risk; (2) fail to focus attention on the highest sites within the oral cavity; (3) tend to group all dysplasias together without regard to the difference of molecular profile; (4) Lack of the commitment and motivation of the examiners (clinicians and dentists), which is a significant factor. Some clinicians consistently find early asymptomatic cancers while others examining the same patient population do not.

In the mean time, few percentages of clinicians and dentists fail to ask some critical questions during examination to help identify the high-risk individuals. For example, only 35% of US dentists ask their patients whether they smoke and fewer (15%) ask about the smokeless tobacco. Correspondingly, nearly 25% of the US and Canadian dental schools do not include any questions on tobacco and alcohol use and 37% do not include both tobacco and alcohol questions during examination procedures. Another survey from Maguire shows that 64% of dentists were unaware of their patients' tobacco habits and 40% did not know their alcohol habits. Failing to identify the high-risk individual and high-risk behaviors led to the difficulty and inefficiency to detect the oral cancer at early stage.

Various procedures have been previously documented for screening patients for oral epithelial cancer using toluidine blue. For example, the U.S. Pat. No. 4,321,251 to Mashberg discloses such a procedure, and U.S. Pat. No. 6,417,003 to Cipriani discloses another similar procedure with more organized form for convenience of routine screening. But both of the methods use the same rinse-application-rinse procedure without identifying the high-risk individuals and high-risk sites, and in general it will results high false positive rate. That is the main reason that FDI World Dental Federation wouldn't recommend to use this method for general population screening. Also, the methods described by Mashberg and Cipriani only vaguely disclaimed the application of Toluidine Blue for screening cancerous and precancerous oral lesions, instead of detecting more specifically high-risk oral tissues due to chromosome allelic loss (or LOH) as described in my this invention.

It is desirable to establish a standard diagnosis procedure which should take account into all above concerns, to screen the lesions with high-risk molecular profiles, identify the high-risk individuals, focus on high-risk sites, reduce false positive and avoid some examination errors.

### BRIEF DESCRIPTION OF THE INVENTION

Briefly, in accordance with the invention, I provide methods and improved procedures of screening patients, using an oral staining agent (toluidine blue) as a tool, for high risk oral tissue which due to chromosome allelic loss (or, Loss of Heterozygosity, LOH) and may have developed or will potentially develop into carcinoma in situ and carcinoma.

The method includes a kit that can be used for a broader application to screen patients for high risk molecular profile for hyperplasia, dysplasia, erythroplasia, leukoplasia, leukoerythroplasia, erythroleukoplasia, verrucous lesion, ulcerative lesion, carcinoma in situ, and carcinoma.

In accordance with one embodiment of the invention I provide an easy-to-use packaged diagnostic kit for performing routine screening procedure to detect high-risk molecular profile that may have developed or potentially develop into carcinoma in situ and carcinoma. The improved procedure includes the steps of sequentially applying preselected volumes with two testing reagents of preselected concentrations to suspected oral lesion sites, with 2 or 3 or more repeats to reduce the false positive.

In accordance with another embodiment of the invention, the kit includes a questionnaire sheet to identify the high-risk individuals and reduce the false positive. Since identifying high-risk individuals or high-risk behaviors would significantly rule out the false positive, the questionnaire would help achieve a more accurate diagnosis for screening high-risk oral tissues, some of which even appear normal.

In accordance with yet another embodiment of the invention, the kit also includes a recording sheet, which will help clinician to focus on high-risk sites and easily and accurately record the result, to avoid some examination errors, also reduce the false positive which due to the examination error in screening patients for high risk oral tissue. Since the repeated examination would be common for many screenings, it would be idea to record the previous results accurately to compare with the results later examined at different time periods. Otherwise, examination errors may occur, either missing the high-risk lesion or including wrong one, especially for those patients who had multiple sites of oral lesions.

Using this improved method and procedure, combined with questionnaire and recording sheet, the false positive can be as low as 2%, if performed well. To reduce the false negative, an occasional equivocal circumscribed light-blue stain should be considered positive.

Also, the kit with packaged form provides convenience for clinician to perform office routine screening by compose only two bottles (rinse and stain solution), and the disposable cups with 10 cc indicator line for easy and accurate dispensing.

If oral or oropharyngeal cancer is identified, further diagnosis should be performed to check the larynx, hypo pharynx, esophagus, and lungs to rule out multiple primary cancers. The patient should also have yearly routine checks.

### DESCRIPTION FOR DRAWINGS

DRAWING. 1: The questionnaire to help reduce false positive. The Key questions are most important questions to identify the high-risk individuals. Optional questions are either minor factor to determine high-risk individuals or to address the unique risk factors for specific populations. For example, in most of Southeast Asia countries, many local people have a habit to chew Betel nut (also known as Pinang or Areca nut), which will significantly increase the risk to have oral cancer. So, it is appropriate to add such an optional question when doing examination at Southeast Asia area.

DRAWING. 2: The recording sheet has pictures that can clearly locate the staining result more accurately than pure description. This will better record the examination result to reduce the examination errors, since the examination usually will be repeated and the results obtained at different time will be compared.

DRAWING. 3: Kit of all items in one box 10, including one big bottle of rinse solution 11 and one small bottle of staining solution 12; disposable cups 13 with volume indicator line; and document bags 14 which can hold the questionnaire sheet, the recording sheet and other necessary documents.

What is claimed is:

1. A diagnostic method of detecting high risk oral tissue which have chromosome allelic loss and may have developed or potentially develop into carcinoma in situ and carcinoma by using toluidine blue staining solution, wherein said staining solution is an aqueous glacial acetic acid solution containing a 1% toluidine blue, also containing but not limiting to ethyl alcohol, hydrogen peroxide, flavoring additives and water, and by including steps of sequentially rinsing the oral cavity with the staining solution, and rinsing the oral cavity with a rinse solution to remove unstained toluidine blue, wherein said rinse solution mainly contains of glacial acid, and also contains but not limits to ethyl alcohol, sodium benzoate, flavoring additives and water, with the detailed examination procedures as following:
(a) rinsing the mouth of the patient with rinse solution for a period of about 20 seconds followed by a similar rinse with water twice for about 20 seconds;
(b) rinsing the mouth of the same patient with 10 cc. staining solution for about 1 minute while simultaneously gargling; and
(c) rinsing with 10 cc rinse solution for about 1 minute followed by a second rinse with rinse solution for 20 seconds, and followed a water rinse.
(d) performing a second repeat in 10-20 days (preferred 15 days), followed with a biopsy if both results are positive, or followed with no biopsy if not double positive but with a third repeat in 3-6 months (preferred 4 months).

2. An examination kit including questionnaire that is used to identify high-risk individuals and reduce the false positive during diagnosis, said questionnaire comprising following key questions with other optional questions:
(a) Have you had any neck and head cancer history before?
(b) Have you had heavy smoking history (more than 10 cigarettes per day, or use smokeless tobacco, cigars or pipes)?
(c) Have you had heavy drinking history (more than three whiskey equivalents per day)?
(d) Have you had oral ulcer for continuous 6 months, or persistent erythroplastic lesions or erythroplakia within a leukoplasia?
(e) Does the lesion occur on the floor of the month, or the ventrolateral tongue, or the soft palate complex?
Said optional questions used to address the minor risk-factors or specificity for the screened population can be exampled but not limited to followings:
1) Do you have habit to chew Betel nut (or Beetal nut, or Pinang, or Areca nut, or Paan, or Gutkha)?
2) Are you older than 40?
3) Do you notice any white, red, or dark lesions anywhere in the mouth?
4) Do you have adequate oral and personal hygiene?
5) Do you have oral habits such as cheek or lip biting?
6) Do you have repeated or unusual bleeding anywhere in the mouth?
7) Do you have difficulty swallowing or chewing?
8) Do you have any swelling, lumps or bumps in mouth?
9) Do you wearold or ill-fitting dentures or have chronic irritation from dentures?
10) Do you have vitamin deficienty, or eat hot or spicy foods, or sharp teeth?
11) Do you have family history of cancer?.

3. An examination kit including a recording sheet with oral cavity diagram to better record the location of stained lesion, said diagram can accurately locate the stained lesions within oral cavity, which can assist clinician to make correct recording for the stained result to avoid such examination error thereof, said recording will be used to compare the repeated staining results to get a better diagnosis.

4. An examination kit in one box, including two bottles of staining solution and rinse solution; disposable cups with volume indicator line; the questionnaire and the recording sheet; and other necessary documents, to make clinician easier to perform screen test.
